(19) United States
(12) Patent Application Publication
(10) Pub. No.: US 2019/0001116 A1
(43) Pub. Date: Jan. 3, 2019

(54) VALVE ARRANGEMENT FOR ENTERAL FEEDING SETS HAVING MULTIPLE FLUID SOURCES

(71) Applicant: AXIUM MTECH SA, Nyon (CH)

(72) Inventors: Michael Jedwab, Lausanne (CH); Nicolas Andrey, Echandens (CH)

(21) Appl. No.: 16/063,014

(22) PCT Filed: Dec. 12, 2016
(86) PCT No.: PCT/EP2016/080574

§ 371 Date: Jun. 15, 2018

Related U.S. Application Data
(60) Provisional application No. 62/268,178, filed on Dec. 16, 2015.

(51) Int. Cl. A61M 39/24; A61M 5/168

(52) U.S. Cl. CPC A61M 39/24; A61M 5/16827

(57) ABSTRACT

A flow set comprises one-way valves and enables the sequential administration of two fluids from a pair of containers to a patient. The predetermined cracking (threshold) pressure of the one-way valves, namely an inlet pressure at which the first indication of flow occurs,can be individually set at predetermined values to minimize and/or prevent free-flow of the two fluids. A connecting tube segment is part of both the flow path of the first fluid and the flow path of the second fluid, and a pump can be connected to the connecting tubing segment. By reversing the pumping direction of the pump, the two fluids may be sequentially drawn from the pair of containers and thus sequentially administered to a patient.

### BACKGROUND

The present disclosure relates generally to a system configured to sequentially administer fluids to a patient, such as medicinal or nutritional fluids in a clinical setting. The fluids may be administered enterally or parenterally. The present disclosure also relates to methods of sequentially administering fluids to a patient.

When a patient is unable to eat normally, an infusion set can provide an enteral solution containing nutrition and medication to the patient. The infusion set can use a pump (e.g., a peristaltic pump) to regulate the amount and the rate at which the enteral solution is delivered from a reservoir to the patient.

Nutritional needs and hydration needs of patients fed enterally are often larger than the regular volume offered in commercially available enteral solutions. To save time, caregivers such as nurses utilize administration sets that allow simultaneous hanging of two separate containers. Individually controlling the flow from each of these containers either adds complexity to the feeding pump or requires additional tubing length that can be tangled or kinked when used by mobile patients in a backpack.

Typically the amount of enteral solution administered to the patient must be precisely controlled, especially if the enteral solution contains potent compounds. In many enteral feeding systems, the engagement of the tube to a peristaltic pump controls the flow of fluid to the patient according to the speed of the peristaltic pump. Nevertheless, excess fluid can reach the patient due to gravity, which is known as free-flow and is not only undesirable but can be dangerous.

Clamps on each of the lines between the fluid container and the pump can allow manual selection of the fluid source. However, a drawback of this configuration is that the user must manually switch from one fluid source to the other fluid source. In most arrangements, this manual switching does not protect against free-flow of the enteral solution.

Two separated pump mechanisms can be used. For example, a delivery set can include a pump interface on both lines between the fluid container and a point of junction into one single line. A drawback of this system is that two independent pumping mechanisms on the pump make the system too heavy and bulky for mobile use.

### SUMMARY

The present disclosure provides a flow set permitting simple and safe switching between two containers to which the flow set is connected. The flow set preferably comprises a first valve assembly separate from a second valve assembly such that the valve assemblies do not share any walls with each other. In this preferred embodiment, a first fluid may be drawn from a first container into the first valve assembly and out through a pump communication port of the first valve assembly, then into the second valve assembly through a pump communication port of the second valve assembly. The first fluid then may flow out of an outlet port of the second valve assembly to the patient. During pumping of the first fluid through the first and second valve assemblies, a one-way valve in the second assembly is closed to prevent the second fluid from moving through the assemblies.

Then, by merely reversing the pumping direction of a pump connected to the flow set, a second fluid may be drawn from a second container through the second valve assembly into the first valve assembly and then to the patient in a manner mirroring the first. During pumping of the second fluid through the first and second valve assemblies, a one-way valve in the first assembly is closed to prevent the first fluid from moving through the assemblies. Preferably, stopping the pump ceases flow in both the first and second flow paths.

No disconnection of tubing is required and no manual adjustment of valves is required. Cracking pressures of flexible membranes in the first and second valve assemblies can be set at predetermined values to minimize and/or prevent free-flow of the first and second fluids.

Accordingly, in a general embodiment, the present disclosure provides a flow set comprising: a first one-way valve, a second one-way valve, a third one-way valve, and a fourth one-way valve. The first one-way valve and the third one-way valve form at least a portion of a first flow path. The second one-way valve and the fourth one-way valve form at least a portion of a second flow path. At least one of the first, second, third and fourth one-way valves has a zero cracking pressure. At least one of the first and third one-way valves has a cracking pressure greater than zero bar, and at least one of the second and fourth one-way valves has a cracking pressure greater than zero bar.

In some embodiments, the first, second, third and fourth one-way valves can have a set of cracking pressures selected from the group consisting of: (i) each of the first and fourth one-way valves has a zero cracking pressure, and each of the second and third one-way valves has a cracking pressure greater than zero bar, (ii) each of the first and fourth one-way valves has a cracking pressure greater than zero bar, and each of the second and third one-way valves has a zero cracking pressure, (iii) each of the first and second one-way valves has a cracking pressure greater than zero bar, and each of the third and fourth one-way valves has a zero cracking pressure, and (iv) each of the third and fourth one-way valves has a cracking pressure greater than zero bar, and each of the first and second one-way valves has a zero cracking pressure, and (v) only one of the first, second, third and fourth one-way valves has a zero cracking pressure, and three of the first, second, third and fourth one-way valves have a cracking pressure greater than zero bar.

In another embodiment, the present disclosure provides a flow set comprising: a first one-way valve, a second one-way valve, a third one-way valve, a fourth one-way valve, a fifth one-way valve upstream of the first one-way valve, and a sixth one-way valve upstream of the fourth one-way valve. The fifth one-way valve, the first one-way valve, and the third one-way valve form at least a portion of a first flow path. The sixth one-way valve, the second one-way valve, and the fourth one-way valve form at least a portion of a second flow path. Each of the fifth and sixth one-way valves has a cracking pressure greater than zero bar. One or more of the first, second, third and fourth one-way valves can have a cracking pressure of zero bar, preferably all four of these one-way valves.

In another embodiment, the present disclosure provides a flow set comprising: a first one-way valve, a second one-way valve, a third one-way valve, a fourth one-way valve, and an additional one-way valve downstream of the second and third one-way valves. The first one-way valve, the third one-way valve and the additional one-way valve form at least a portion of a first flow path. The second one-way valve, the fourth one-way valve and the additional one-way valve form at least a portion of a second flow path. The additional one-way valve has a cracking pressure greater than zero bar. One or more of the first, second, third and fourth one-way valves can have a cracking pressure of zero bar, preferably all four of these one-way valves.

In each of these embodiments, preferably each of the first, second, third and fourth one-way valves is a slit valve. A first valve assembly can provide the first and second one-way valves, and a second valve assembly can provide the third and fourth one-way valves.

The present disclosure also provides methods of using and making such flow sets.

An advantage of one or more embodiments provided by the present disclosure is to automatically select a fluid container of an enteral administration set having a plurality of fluid containers.

Another advantage of one or more embodiments provided by the present disclosure is an enteral administration set having a plurality of fluid containers that uses one single pumping mechanism.

A further advantage of one or more embodiments provided by the present disclosure is to reduce pump complexity by eliminating the requirement for an additional actuator to select the fluid container.

Yet another advantage of one or more embodiments provided by the present disclosure is to increase safety by preventing free-flow situations.

Additional features and advantages are described herein and will be apparent from the following Detailed Description and the Figures.

### BRIEF DESCRIPTION OF THE FIGURES

FIG. 1 illustrates an embodiment of a flow set provided by the present disclosure, connected to two containers.

FIG. 2 illustrates an embodiment of a system provided by the present disclosure, connected to two containers.

FIG. 3A is a cross-sectional view of an embodiment of a valve assembly used in the systems of FIGS. 1 and 2 in a rest state.

FIG. 3B is a cross-sectional view of the valve assembly of FIG. 3A in a first operative state.

FIG. 3C is a cross-sectional view of the valve assembly of FIGS. 3A and 3B in a second operational state.

FIG. 4 is a schematic illustration of fluid flow in the system of FIG. 2.

### DETAILED DESCRIPTION

As used in this disclosure and the appended claims, the singular forms “a,” “an” and “the” include plural referents unless the context clearly dictates otherwise. Thus, for example, reference to “a fluid” or “the fluid” includes two or more fluids.

The words “comprise,” “comprises” and “comprising” are to be interpreted inclusively rather than exclusively. Likewise, the terms “include,” “including” and “or” should all be construed to be inclusive, unless such a construction is clearly prohibited from the context.

Nevertheless, the devices and apparatuses disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

As used herein, “about” and “approximately” are understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number.

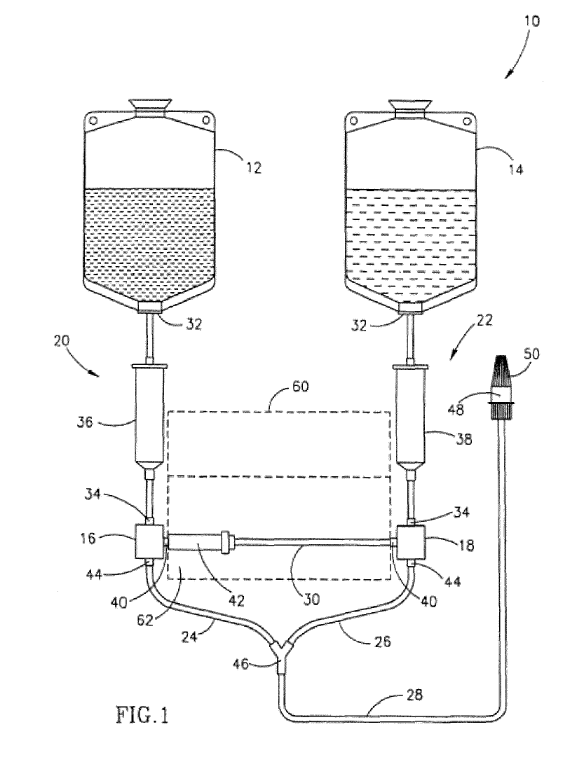

As shown in FIG. 1, an aspect of the present disclosure is a disposable flow set 10 which may be used to sequentially administer a first fluid (e.g., a first enteral feeding formulation) and a second fluid (e.g., a second enteral feeding formulation). The flow set 10 can comprise a first container 12 that holds the first fluid, a second container 14 that holds the second fluid, a first valve assembly 16, and a second valve assembly 18. In an embodiment, the first container 12 is connected to a first drip chamber 36, and/or the second container 14 is connected to a second drip chamber 38.

In a preferred embodiment, the first valve assembly 16 is a single unitary device, and the second valve assembly 18 is another single unitary device. However, in other embodiments the first valve assembly 16 is a plurality of separate devices, such as a pair of valves separate from each other in different housings, and the second valve assembly 18 is another plurality of separate devices, such as another pair of valves separate from each other in different housings.

Each of the first and second containers 12, 14 can comprise an outlet 32. Each of the first and second valve assemblies 16, 18 can comprise an inlet port 34 and an outlet port 44. Each of the first and second valve assemblies 16, 18 can use any number of inlet ports and outlet ports, and the present disclosure is not limited to a specific number of inlet ports and outlet ports.

A connecting tube 30 can connect the first valve assembly 16 and the second valve assembly 18 to each other. For example, the first valve assembly 16 and the second valve assembly 18 can be distanced from each other by the connecting tube 30 extending therebetween. The connecting tube 30 can span between the first and second valve assemblies 16, 18. Opposite ends of the connecting tube 30 can connect to a pump communication port 40 of each of the first and second valve assemblies 16, 18. A connecting element 42 can be coupled to the connecting tube 30.

The flow set 10 can comprise a first tube segment 20. One end of the first tube segment 20 can be connected to the outlet 32 of the first container 12, and the other end of the first tube segment 20 can be connected to the inlet port 34 of the first valve assembly 16. The first drip chamber 36 can be coupled to the first tube segment 20.

The flow set 10 can comprise a second tube segment 22. One end of the second tube segment 22 can be connected to the outlet 32 of the second container 14, and the other end of the second tube segment 22 can be connected to the inlet port 34 of the second valve assembly 18. The second drip chamber 38 can be coupled to the second tube segment 22.

The flow set 10 can comprise a third tube segment 24. One end of the third tube segment 24 can be connected to the outlet port 44 of the first valve assembly 16, and the other end can be connected to a Y-connector 46.

The flow set 10 can comprise a fourth tube segment 26. One end of the fourth tube segment 26 can be connected to the outlet port 44 of the second valve assembly 18, and the other end can be connected to the Y-connector 46.

Each of the first, second, third, fourth and connecting tubes 20, 22, 24, 26, 30 can be made of a flexible material such as polyvinyl chloride or silicone rubber. In some embodiments, one or more of the first, second, third, fourth and connecting tubes 20, 22, 24, 26, 30 are part of the first valve assembly 16 and/or the second valve assembly 18.

The Y-connector 46 can connect the third and fourth tube segments 24, 26 to an administration tube 28. The Y-connector 46 is preferably positioned downstream of the connecting tube 30. The free end of the administration tube 28 can be connected to a connector 48 which may be connected to a catheter, an enteral feeding tube, or another device configured to administer a fluid to a patient. The free end of the connector 48 can be closed by a cover 50 when the flow set 10 is not in use.

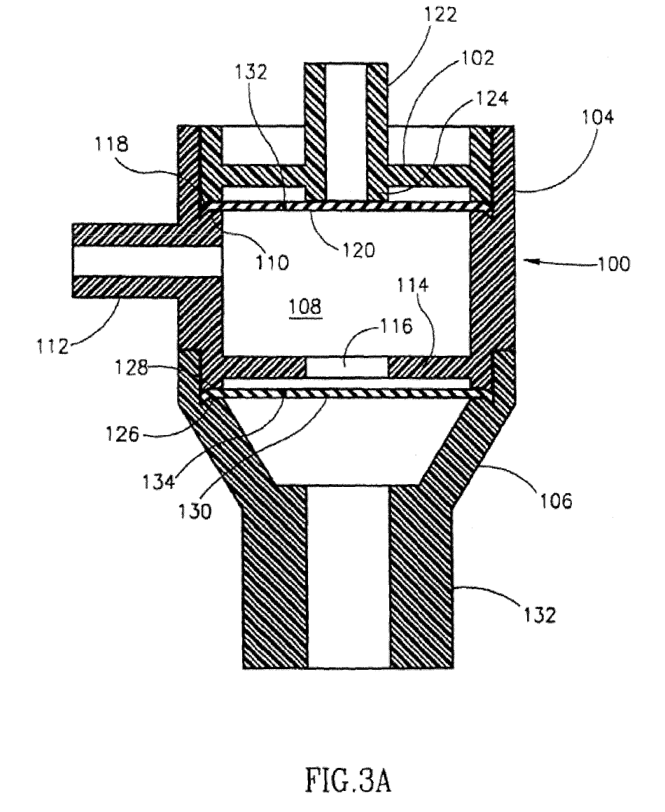

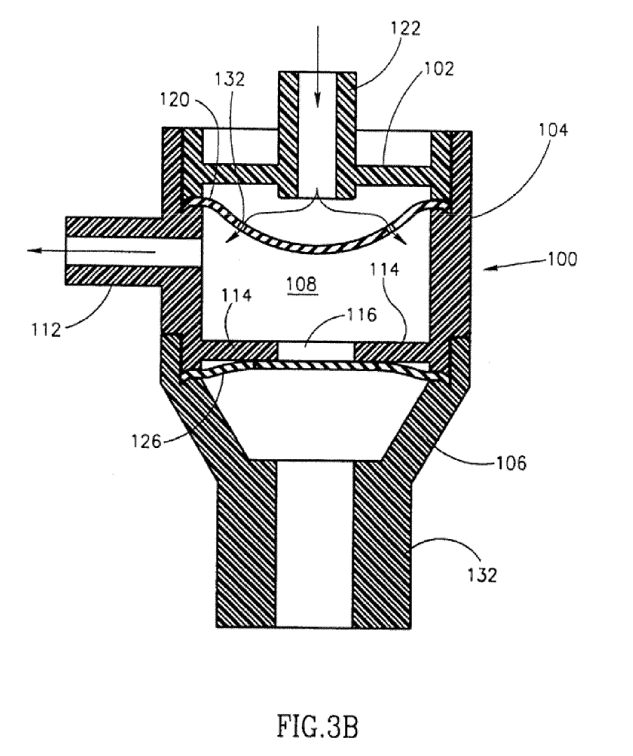

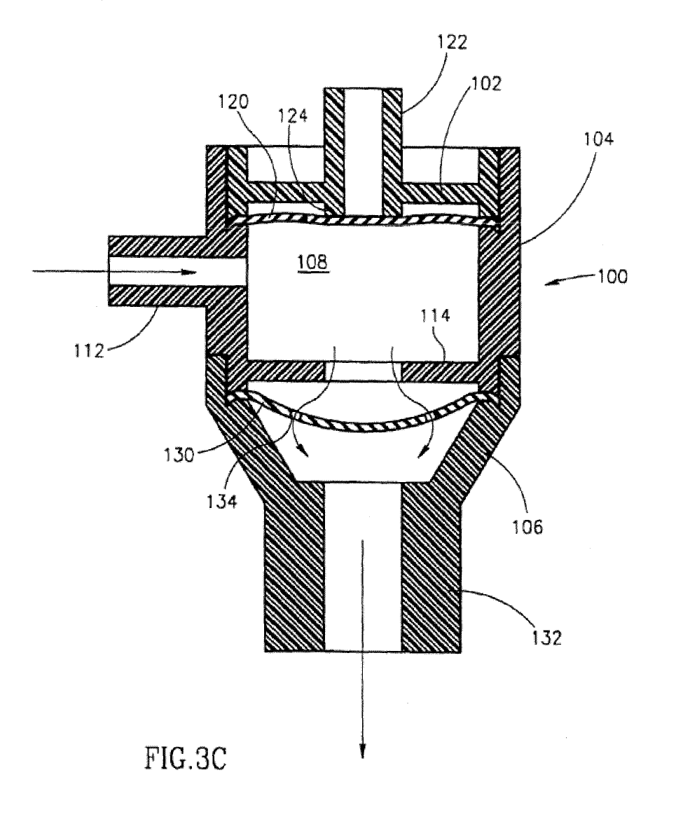

As shown in FIG. 3A, each of the first and second valve assemblies 16, 18 can comprise a housing 100 comprising a first body member 102, a second body member 104 and a third body member 106. The housing 100 can be made of a metal and/or a plastic, such as acrylonitrile butadiene styrene (ABS), polycarbonate, polyvinyl chloride (PVC), an acrylic material, or methyl methacrylate-acrylonitrile-butadiene-styrene (MABS).

The second body member 104 can be a tube comprising a lower end that is closed by a base plate 114. The base plate 114 can have an opening 116 therethrough. The second body member 104 can comprise a central bore that can form a chamber 108 positioned above the base plate 114 such that the opening 116 forms an outlet of the chamber 108. The second body member 104 preferably comprises an annular shoulder 110 formed in the inner side walls of the second body member 104, adjacent the upper end of the second body member 104. A lateral port 112 can extend through the side walls of the second body member 104, between the annular shoulder 110 and the base plate 114. The lateral port 112 can form the pump communication port 40 to which the connecting tube 30 can connect.

At least a portion of the first body member 102 can have the same shape and the same size as a portion of the central bore of the second body member 104. As a result, the first body member 102 can insert into the central bore of the second body member 104 (e.g., by friction fit) to form an upper seal for the chamber 108.

The first body member 102 can comprise a first annular tab 118 projecting away (e.g., downward) from the lower end of the first body member 102. The first annular tab 118 preferably has a shape is complementary to the annular shoulder 110 of the second body member 104 such that the annular shoulder 110 and the annular tab 118 form an annular clamp when the first body member 102 is fitted into the second body member 104. An inlet tube 122 can extend through the first body member 102 to define the inlet port 34 to the valve assembly 16, 18. The first body member 102 can have an annular rim 124 projecting into the chamber 108 about the inlet tube 122. In an embodiment, a first flexible membrane 120 is clamped between the annular shoulder 110 and the annular tab 118.

The third body member 106 can be a tube having the shape of a funnel. An annular shoulder 126 can be defined in the side walls of a central bore of the third body member 106 adjacent the upper end of the third body member 106.

The base plate 114 can comprise a second annular tab 128 projecting away (e.g., downward) from the lower end of the base plate 114. The annular shoulder 126 preferably has a shape complementary to the lower end of the second annular tab 128 such that the annular tab 128 and the annular shoulder 126 form an annular clamp when the second body member 104 is fitted onto the third body member 106.

At least a portion of the second annular tab 128 can have the same shape and the same size as a portion of the central bore of the third body member 106. As a result, the second annular tab 128 of the second body member 104 can insert into the central bore of the third body member 106 (e.g., by friction fit) to seal the upper end of the third body member 106.

An outlet tube 132 can extend from the third body member 106 to define the outlet port 44 from the valve assembly 16, 18. In an embodiment, a second flexible membrane 130 is clamped between the second annular tab 128 of the second body member 104 and the annular shoulder 126 of the third body member 106.

Each of the first and second flexible membranes 120, 130 can be made of a resilient flexible material, preferably a sterilizable material such as silicon, rubber or any suitable material. In an embodiment, the first and second flexible membranes 120, 130 each have slits 132, 134 (two slits shown in each membrane, but any number can be used). When the first and second flexible membranes 120, 130 are in the resting state shown in FIG. 3A, the slits 132, 134 are completely closed and do not permit flow of fluid through the corresponding one of the first and second flexible membranes 120, 130. Therefore, the first flexible membrane 120 of the first valve assembly 16 can function as a first one-way valve, the second flexible membrane 130 of the first valve assembly 16 can function as a second one-way valve, the second flexible membrane 130 of the second valve assembly 18 can function as a third one-way valve, and the first flexible membrane 120 of the second valve assembly 18 can function as a fourth one-way valve, as discussed in more detail later herein.

Each of the first flexible membrane 120 of the first valve assembly 16, the second flexible membrane 130 of the first valve assembly 16, the first flexible membrane 120 of the second valve assembly 18, and the second flexible membrane 130 of the second valve assembly 18 can have a predetermined cracking (threshold) pressure, namely an inlet pressure at which the first indication of flow occurs. For example, the cracking pressures of the first flexible membrane 120 of the first valve assembly 16, the second flexible membrane 130 of the first valve assembly 16, the first flexible membrane 120 of the second valve assembly 18, and the second flexible membrane 130 of the second valve assembly 18 can be set at predetermined values to minimize and/or prevent free-flow of the first and second fluids.

In one such embodiment, both of the first flexible membrane 120 of the first valve assembly 16 (the first one-way valve) and the first flexible membrane 120 of the second valve assembly 18 (the fourth one-way valve) have a zero cracking pressure, and both of the second flexible membrane 130 of the first valve assembly 16 (the second one-way valve) and the second flexible membrane 130 of the second valve assembly 18 (the third one-way valve) have a cracking pressure greater than zero bar (e.g., about 0.1 bar to about 10.0 bar, such as about 0.3 bar).

In another such embodiment, both of the first flexible membrane 120 of the first valve assembly 16 (the first one-way valve) and the first flexible membrane 120 of the second valve assembly 18 (the fourth one-way valve) have a cracking pressure greater than zero bar (e.g., about 0.1 bar to about 10.0 bar, such as about 0.3 bar), and both of the second flexible membrane 130 of the first valve assembly 16 (the second one-way valve) and the second flexible membrane 130 of the second valve assembly 18 (the third one-way valve) have a zero cracking pressure.

In yet another such embodiment, both of the first flexible membrane 120 of the first valve assembly 16 (the first one-way valve) and the second flexible membrane 130 of the first valve assembly 16 (the second one-way valve) have a cracking pressure greater than zero bar (e.g., about 0.1 bar to about 10.0 bar, such as about 0.3 bar), and both of the second flexible membrane 130 of the second valve assembly 18 (the third one-way valve) and the first flexible membrane 120 of the second valve assembly 18 (the fourth one-way valve) have a zero cracking pressure.

In yet another such embodiment, both of the first flexible membrane 120 of the first valve assembly 16 (the first one-way valve) and the second flexible membrane 130 of the first valve assembly 16 (the second one-way valve) have a zero cracking pressure, and both of the second flexible membrane 130 of the second valve assembly 18 (the third one-way valve) and the first flexible membrane 120 of the second valve assembly 18 (the fourth one-way valve) have a cracking pressure greater than zero bar (e.g., about 0.1 bar to about 10.0 bar, such as about 0.3 bar).

In yet another such embodiment, only one of the first flexible membrane 120 of the first valve assembly 16 (the first one-way valve), the second flexible membrane 130 of the first valve assembly 16 (the second one-way valve), the second flexible membrane 130 of the second valve assembly 18 (the third one-way valve), or the first flexible membrane 120 of the second valve assembly 18 (the fourth one-way valve) has a zero cracking pressure. The other three of these one-way valves each have a cracking pressure greater than zero bar (e.g., about 0.1 bar to about 10.0 bar, such as about 0.3 bar).

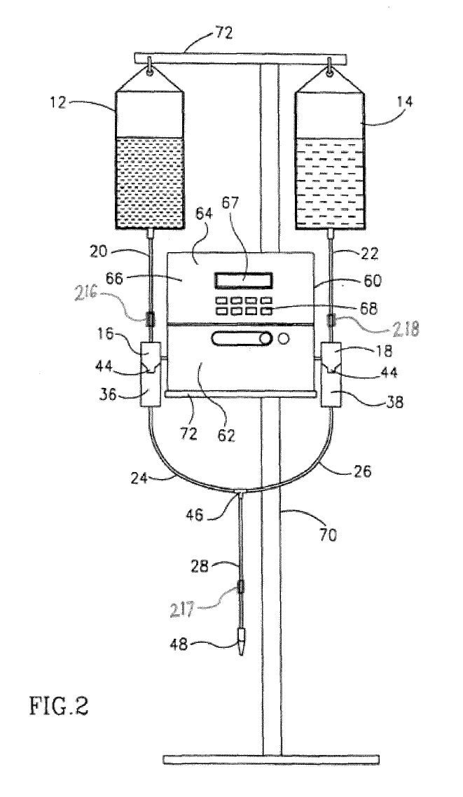

Some embodiments of the flow set 10 comprise an anti-free flow mechanism comprising one or more additional one-way valves. For example, as shown in FIG. 2, a fifth one-way valve 216 and a sixth one-way valve 218 can be positioned upstream from the first and second valve assemblies 16, 18. Additionally or alternatively, a seventh one-way valve 217 can be positioned downstream from the first and second valve assemblies 16, 18 (e.g., downstream from the Y-connector 46).

In these embodiments, at least one of the first, second, third and fourth one-way valves (e.g., one, two, three or all four of these one-way valves) can have a zero cracking pressure or a very low cracking pressure (e.g., less than about 0.1 bars), and the one or more one-way valves in the separate anti-free flow mechanism have a cracking pressure greater than zero bar (e.g., about 0.1 bar to about 10.0 bar, such as about 0.3 bar). For example, the fifth and sixth one-way valves 216, 218 can be present and can have a cracking pressure greater than zero bar (e.g., about 0.1 bar to about 10.0 bar, such as about 0.3 bar). As another example, the seventh one-way valve 217 can be present and can have a cracking pressure greater than zero bar (e.g., about 0.1 bar to about 10.0 bar, such as about 0.3 bar).

The fifth one-way valve 216 can be associated with the first container 12 and/or the first connecting tube 20, and the sixth one-way valve 218 can be associated with the second container 14 and/or the second connecting tube 22. In some embodiments, the fifth and sixth one-way valves 216, 218 are part of the first and second valve assemblies 16, 18 respectively; in other embodiments, the fifth and sixth one-way valves 13f, 13g are external to the first and second valve assemblies 16, 18. Preferably the separate anti-free flow mechanism, if present, only comprises one of (i) the fifth and sixth one-way valves 216, 218 or (ii) the seventh one-way valve 217; but in some embodiments all three of the fifth, sixth and seventh one-way valves 216-218 are present.

The cracking pressure threshold of each of the first flexible membrane 120 of the first valve assembly 16, the second flexible membrane 130 of the first valve assembly 16, the first flexible membrane 120 of the second valve assembly 18, and the second flexible membrane 130 of the second valve assembly 18 can be selectively established when the flow set 10 is made. For example, the threshold can be established by selecting one or more of: a thickness of the flexible membrane, the surface area of the flexible membrane, and the geometry of the slits (e.g., size and/or length) in the flexible membrane.

As shown in FIGS. 1 and 2, a pump unit 60 comprising a pump 62 can be coupled to the connecting tube 30 of the flow set 10. The pump 62 is preferably a rotating peristaltic pump but can be any pump able to pump fluid at controlled flow rates in both directions and suitable for clinical applications. The present disclosure is not limited to a specific embodiment of the pump 62, and the pump 62 can operate using a mechanism alternative or additional to rotation.

As shown in FIG. 2, the pump unit 60 may comprise a control unit 64 that can comprise a control panel 66 comprising a display 67 and/or a key pad 68. The key pad 68 may be used for manual control of the pump 62, data entry, and the like. The control unit 64 can comprise a microprocessor configured to control and activate the pump 62. A memory may be associated with and/or be incorporated in the microprocessor. The control unit 64 may provide an audio, visual or dual alarm.

The pump unit 60 can comprise a socket complementary to the connecting element 42. In such an embodiment, the connecting element 42 fits into the socket in the pump unit 60 when the system comprising the pump unit 60 and the flow set 10 is correctly assembled. The pump unit 60 may be provided with a microswitch in the socket which generates a signal when the connecting element 42 is positioned in the socket. This signal can identify to the control unit 64 that the system has been correctly assembled. The control unit 64 may be programmed not to initiate the pump 62 unless the signal has been received.

The pump 62 is configured to provide suction to the chamber 108 of the first valve assembly 16 while simultaneously discharging into the chamber 108 of the second valve assembly 18. Similarly, the pump 62 is configured to provide suction to the chamber 108 of the second valve assembly 18 while simultaneously discharging into the chamber 108 of the first valve assembly 16.

As generally illustrated in FIG. 2, the flow set 10 can be mounted on a stand 70 with the first and second containers 12, 14 held by an arm 72 at the top of the stand 70. The embodiment of the pump system shown schematically in FIG. 2 differs from the embodiment of FIG. 1 in that the drip chambers 36, 38 are coupled to the outlet ports 44 of the first and second valve assemblies 16, 18 in the embodiment in FIG. 2 and not positioned in the first and second tube segments 20, 22.

In either embodiment, the pump 62 is initiated to pump fluid from one of the first and second containers 12, 14 to a patient. For example, the pump 62 can draw the first fluid from the first container 12. The first fluid can be drawn into the first tube segment 20, then through the drip chamber 36, and then into the inlet port 34 of the first valve assembly 16.

Prior to the initiation of the pump 62, the first valve assembly 16 is in the rest state illustrated in FIG. 3A. When the first fluid is drawn through the inlet tube 122 of the first valve assembly 16, the first flexible membrane 120 of the first valve assembly 16 is stretched and deflected as shown in FIG. 3B. When the selected threshold pressure differential is reached and the first flexible membrane 120 of the first valve assembly 16 is sufficiently stretched, the slits 132 in the first membrane 120 widen and open to allow flow of the first fluid from the inlet tube 122 into the chamber 108 of the first valve assembly 16.

Simultaneously, the suction of the pump 62 reduces the pressure in the connecting tube 30 at the feed end of the pump 62 and in the chamber 108 of the first valve assembly 16. This reduced pressure in the chamber 108 of the first valve assembly 16 causes the second flexible membrane 130 of the first valve assembly 16 to seal against the base plate 114 of the second body member 104. The first fluid entering the chamber 108 then flows from the chamber 108 through the lateral port 112 and into the connecting tube 30. The first fluid is unable to penetrate through the second flexible membrane 130 of the first valve assembly 16.

The first fluid is then propelled by the pump 62 to the second valve assembly 18. As shown in FIG. 3C, the first fluid enters the second valve assembly 18 through the lateral port 112 (i.e., the pump communication port 40) of the second valve assembly 18. The first fluid in the chamber 108 of the second valve assembly 18 forces the first flexible membrane 120 of the second valve assembly 18 against the first body member 102 of the second valve assembly 18. Consequently, the first flexible membrane 120 of the second valve assembly 18 cannot deflect sufficiently to permit fluid flow therethrough. As a result, the first flexible membrane 120 of the second valve assembly 18 prevents the second fluid from flowing into through the second valve assembly 18 while the first fluid is pumped through the first and second valve assemblies 16, 18.

However, the positive pressure induced in the chamber 108 of the second valve assembly 18 by the pump 62 can cause the second flexible membrane 130 of the second valve assembly 18 to stretch and deflect. When the selected threshold pressure differential is reached and the second flexible membrane 130 of the second valve assembly 18 is sufficiently stretched, the slits 134 in the second flexible membrane 130 widen and open to allow flow of the first fluid from the chamber 108 of the second valve assembly 18 out of the outlet tube 132 of the second valve assembly 18.

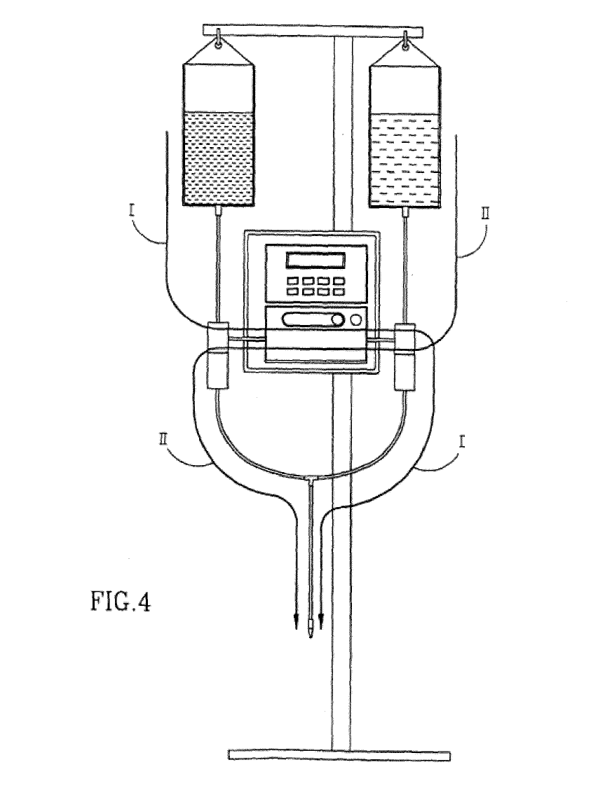

The first fluid then can flow through the fourth tube segment 26 which is connected to the outlet port 44 of the second valve assembly 18, then through the Y-connector 46, and into the administration tube 28. A small amount of the first fluid may initially flow into the third tube segment 24 but is prevented from entering the first valve assembly 16 by the second flexible membrane 130 of the first valve assembly 16. Therefore, the flow path of the first fluid is as illustrated by arrow I in FIG. 4.

When administration of the second fluid is desired, the pumping direction of the pump 62 is reversed. The reversal of pumping direction draws the second fluid through the inlet tube 122 of the second valve assembly 18 and stretches and deflects the first flexible membrane 120 of the second valve assembly 18. When the selected threshold pressure differential is reached and the first flexible membrane 120 of the second valve assembly 18 is sufficiently stretched, the slits 132 in the first membrane 120 widen and open to allow flow of the second fluid from the inlet tube 122 into the chamber 108 of the second valve assembly 18.

Simultaneously, the suction of the pump 62 reduces the pressure in the connecting tube 30 at the feed end of the pump 62 and in the chamber 108 of the second valve assembly 18. This reduced pressure in the chamber 108 of the second valve assembly 18 causes the second flexible membrane 130 of the second valve assembly 18 to seal against the base plate 114 of the second body member 104. The second fluid entering the chamber 108 then flows from the chamber 108 through the lateral port 112 and into the connecting tube 30. The second fluid is unable to penetrate through the second flexible membrane 130 of the second valve assembly 18.

The second fluid is then propelled by the pump 62 to the first valve assembly 16. The second fluid enters the first valve assembly 16 through the lateral port 112 (i.e., the pump communication port 40) of the first valve assembly 16. The second fluid in the chamber 108 of the first valve assembly 16 forces the first flexible membrane 120 of the first valve assembly 16 against the first body member 102 of the first valve assembly 16. Consequently, the first flexible membrane 120 of the first valve assembly 16 cannot deflect sufficiently to permit fluid flow therethrough. As a result, the first flexible membrane 120 of the first valve assembly 16 prevents the first fluid from flowing into through the first valve assembly 16 while the second fluid is pumped through the first and second valve assemblies 16, 18.

However, the positive pressure induced in the chamber 108 of the first valve assembly 16 by the pump 62 can cause the second flexible membrane 130 of the first valve assembly 16 to stretch and deflect. When the selected threshold pressure differential is reached and the second flexible membrane 130 of the first valve assembly 16 is sufficiently stretched, the slits 134 in the second flexible membrane 130 widen and open to allow flow of the second fluid from the chamber 108 of the first valve assembly 16 out of the outlet tube 132 of the first valve assembly 16.

The second fluid then can flow through the third tube segment 24 which is connected to the outlet port 44 of the first valve assembly 16, then through the Y-connector 46, and into the administration tube 28. A small amount of the second fluid may initially flow into the fourth tube segment 26 but is prevented from entering the second valve assembly 18 by the second flexible membrane 130 of the second valve assembly 18. Therefore, the flow path of the second fluid is illustrated by arrow II in FIG. 4.

The system comprising the pump unit 60 and the flow set 10 thus safely and easily provides sequential administration of the first and second fluids to a patient.

The system may be operated in various modes. For example, the system may initially flush the flow set 10 with a flushing solution and then switch to a feeding fluid. At selected intervals, the system may then switch back to the flushing solution for a short period to flush the flow set 10 to reduce the probability of blockages. As another example, the system may deliver a predetermined amount of one of the first and second fluids to the patient and then deliver a predetermined amount of the other fluid to the patient. As yet another example, the system may intermittently deliver a predetermined amount of one of the first and second fluids to the patient and then deliver a predetermined amount of the other fluid to the patient.

Numerous modifications may be made to the preferred embodiments. For example, the drip chambers 36, 38 do not need to be connected in the flow set 10 or even present in the system. As another example, the pump 62 does not necessarily have a socket, and the connecting tube 30 does not necessarily have the connecting element 42. Similarly, the Y-connector 46 may be replaced with any suitable connector. Similarly, valve arrangements other than the first and second valve assemblies 16, 18 may be used; for example, each of the first and second valve assemblies 16, 18 may be replaced with a pair of one-way valves which open upon a threshold pressure being reached. The connecting tube 30 will then extend from a position between the pair of one-way valves of each of the first and second valve assemblies 16, 18.

Another aspect of the present disclosure is a method of sequentially administering a first fluid and a second fluid to a patient using a single pump connected to a flow set. The method can use the pump unit 60 and the flow set 10 or any pump unit and flow set capable of performing the method. The method preferably utilizes first, second, third and fourth tube segments.

The method can comprise operating the pump in one pumping direction to direct a first fluid from a first container through a first flow path. The first flow path can comprise a first tube segment, which preferably connects the first container to an inlet port of a first valve assembly; then a chamber in the first valve assembly; then a connecting tube extending from a pump communication port of the first valve assembly into a pump communication port of the second valve assembly; then a chamber in the second valve assembly; then a fourth tube segment, which preferably extends from an outlet port of the second valve assembly. Preferably a one-way valve in the second valve assembly (e.g., a first flexible membrane in the second valve assembly) prevents the second fluid from flowing through the flow set while the first fluid is pumped through the flow set.

The method can comprise reversing the pumping direction of the pump to cease flow of the first fluid through the first flow path and direct the second fluid from a second container in a second flow path. The second flow path can comprise a second tube segment, which preferably connects the second container to an inlet port of the second valve assembly; then the chamber in the second valve assembly; then the connecting tube extending from the pump communication port of the second valve assembly into the pump communication port of the first valve assembly; then the chamber in the first valve assembly; then a third tube segment, which preferably extends from an outlet port of the first valve assembly. A one-way valve in the first valve assembly (e.g., a first flexible membrane in the first valve assembly) prevents the second fluid from flowing through the flow set while the first fluid is pumped through the flow set.

The method can comprise stopping the pump to cease flow in both the first and second flow paths.

In an embodiment, the third and fourth tube segments are connected to an administration tube that leads to the patient. For example, the third and fourth tube segments and the administration tube can meet at a Y-connector.

The first flow path can comprise a first one-way valve, preferably provided by the first valve assembly (e.g., a first flexible membrane provided by the first valve assembly), and the first one-way valve can be downstream of the inlet port of the first valve assembly and upstream of the chamber of the first valve assembly. The second flow path can comprise a second one-way valve, preferably provided by the first valve assembly (e.g., a second flexible membrane provided by the first valve assembly), and the second one-way valve can be downstream of the chamber of the first valve assembly and upstream of the outlet of the first valve assembly.

The first flow path can comprise a third one-way valve, preferably provided by the second valve assembly (e.g., a second flexible membrane provided by the second valve assembly), and the third one-way valve can be downstream of the chamber of the second valve assembly and upstream of the outlet port of the second valve assembly. The second flow path can comprise a fourth one-way valve, preferably provided by the second valve assembly (e.g., a first flexible membrane provided by the first valve assembly), and the fourth one-way valve can be downstream of the inlet port of the second valve assembly and upstream of the chamber of the first valve assembly.

The first, second, third and fourth one-way valves can each be slit valves. Nevertheless, each of the first, second, third and fourth one-way valves is not limited to a specific type of valve and may be any suitable valve known to one skilled in this art.

Preferably, at least one of the first, second, third and fourth one-way valves has a zero cracking pressure; at least one of the first and third one-way valves has a cracking pressure greater than zero bar (e.g., about 0.1 bar to about 10.0 bar, such as about 0.3 bar); and at least one of the second and fourth one-way valves has a cracking pressure greater than zero bar (e.g., about 0.1 bar to about 10.0 bar, such as about 0.3 bar).

In an embodiment, each of the first and fourth one-way valves has a zero cracking pressure, and each of the second and third one-way valves has a cracking pressure greater than zero bar (e.g., about 0.1 bar to about 10.0 bar, such as about 0.3 bar). In another embodiment, each of the first and fourth one-way valves has a cracking pressure greater than zero bar (e.g., about 0.1 bar to about 10.0 bar, such as about 0.3 bar), and each of the second and third one-way valves has a zero cracking pressure. In another embodiment, each of the first and second one-way valves has a cracking pressure greater than zero bar (e.g., about 0.1 bar to about 10.0 bar, such as about 0.3 bar), and each of the third and fourth one-way valves has a zero cracking pressure. In another embodiment, each of the first and second one-way valves has a zero cracking pressure, and each of the third and fourth one-way valves has a cracking pressure greater than zero bar (e.g., about 0.1 bar to about 10.0 bar, such as about 0.3 bar). In another embodiment, only one of the first, second, third and fourth one-way valves has a zero cracking pressure, and the other three of the first, second, third and fourth one-way valves have a cracking pressure greater than zero bar (e.g., about 0.1 bar to about 10.0 bar, such as about 0.3 bar).

In yet another embodiment, a fifth one-way valve upstream from the first one way valve can be used, and a sixth one-way valve upstream from the fourth one-way valve can be used. Each of the fifth and sixth one-way valves has a cracking pressure greater than zero bar (e.g., about 0.1 bar to about 10.0 bar, such as about 0.3 bar). One or more of the first, second, third and fourth one-way valves (e.g., one, two, three or all four of these one-way valves) has a zero cracking pressure.

In yet another embodiment, an additional valve which is downstream of the Y-connector but before the patient can be used. The additional one-way valve has a cracking pressure greater than zero bar (e.g., about 0.1 bar to about 10.0 bar, such as about 0.3 bar). One or more of the first, second, third and fourth one-way valves (e.g., one, two, three or all four of these one-way valves) has a zero cracking pressure.

In each of the first and second valve assemblies, preferably the inlet, outlet and pump communication ports communicate with each other through the chamber.

Preferably the pumping direction of the pump is reversed automatically according to instructions stored within a control unit operatively connected to the pump. The method preferably does not include disconnection of tubing, does not include changing of pumps, and does not include manual adjustment of any valves. In an embodiment, the pumping direction of the pump is reversed at least twice. For example, the method can further comprise reversing the pumping direction of the pump an additional time to cease movement of the second fluid through the second flow path and again direct the first fluid through the first flow path.

Yet another aspect of the present disclosure is a method of making a flow set configured to connect to a single pump and sequentially administer a first fluid and a second fluid to a patient. The method can make the flow set 10 disclosed herein or any delivery device capable of being made by the steps of the method.

The method can comprise forming a first valve assembly that contains a portion of a first flow path and a portion of a second flow path. The first valve assembly comprises a chamber. The first valve assembly comprises an inlet port, a pump communication port, and an outlet port that communicate with each other through the chamber. The portion of the first flow path provided by the first valve assembly extends from the inlet port through the chamber to the pump communication port. The portion of the second flow path provided by the first valve assembly extends from the pump communication port through the chamber to the outlet port.

The method can comprise forming a second valve assembly that contains a portion of the first flow path and a portion of the second flow path. The second valve assembly comprises a chamber. The second valve assembly comprises an inlet port, a pump communication port, and an outlet port that communicate with each other through the chamber. The portion of the second flow path provided by the second valve assembly extends from the inlet port through the chamber to the pump communication port. The portion of the first flow path provided by the first valve assembly extends from the pump communication port through the chamber to the outlet port.

In an embodiment, the method comprises establishing a predetermined cracking pressure for each of the valves. For example, at least one of the first, second, third and fourth one-way valves can have a zero cracking pressure; at least one of the first and third one-way valves can have a cracking pressure greater than zero bar; and at least one of the second and fourth one-way valves can have a cracking pressure greater than zero bar.

In one such embodiment, each of the first and fourth one-way valves has a zero cracking pressure, and each of the second and third one-way valves has a cracking pressure greater than zero bar (e.g., about 0.1 bar to about 10.0 bar, such as about 0.3 bar). In another such embodiment, each of the first and fourth one-way valves has a cracking pressure greater than zero bar (e.g., about 0.1 bar to about 10.0 bar, such as about 0.3 bar), and each of the second and third one-way valves has a zero cracking pressure.

In another such embodiment, each of the first and second one-way valves has a zero cracking pressure, and each of the third and fourth one-way valves has a cracking pressure greater than zero bar (e.g., about 0.1 bar to about 10.0 bar, such as about 0.3 bar). In another such embodiment, each of the first and second one-way valves has a cracking pressure greater than zero bar (e.g., about 0.1 bar to about 10.0 bar, such as about 0.3 bar), and each of the third and fourth one-way valves has a zero cracking pressure.

In another such embodiment, only one of the first, second, third and fourth one-way valves has a zero cracking pressure, and the other three of the first, second, third and fourth one-way valves have a cracking pressure greater than zero bar (e.g., about 0.1 bar to about 10.0 bar, such as about 0.3 bar).

In yet another such embodiment, a fifth one-way valve upstream from the first one way valve can be used, and a sixth one-way valve upstream from the fourth one-way valve can be used. Each of the first, second, third and fourth one-way valves has a zero cracking pressure, and each of the fifth and sixth one-way valves has a cracking pressure greater than zero bar (e.g., about 0.1 bar to about 10.0 bar, such as about 0.3 bar).

In yet another such embodiment, an additional valve which is downstream of the Y-connector but before the patient can be used. Each of the first, second, third and fourth one-way valves has a zero cracking pressure, and the additional one-way valve has a cracking pressure greater than zero bar (e.g., about 0.1 bar to about 10.0 bar, such as about 0.3 bar).

The method can further comprise positioning a first one-way valve, a second one-way valve, a third one-way valve and a fourth one way valve within the first and second valve assemblies such that the first one-way valve and the third one-way valve are positioned in the first flow path, the second one-way valve and the fourth one-way valve are positioned in the second flow path, the first and second one-way valves are positioned in the first valve assembly, and the third and fourth one-way valves are positioned in the first valve assembly.

In an embodiment, each of the first, second, third and fourth one-way valves is a slit valve. Nevertheless, each of the first, second, third and fourth one-way valves is not limited to a specific type of valve and may be any suitable valve known to one skilled in this art.

Yet another aspect of the present disclosure is a method of making a system configured to sequentially administer a first fluid and a second fluid to a patient. The method comprises connecting to a pump any embodiment of a flow set disclosed herein or any flow set made by a method disclosed herein. The pump can be a positive displacement pump. The pump can be connected to the pump communication port of the first valve assembly and the pump communication port of the second valve assembly. The pump can be configured to pump in a first direction and a second direction opposite to the first direction to alternate suction through the pump communication port of the first valve assembly and suction through the pump communication port of the second valve assembly.

The method can comprise connecting the first tube segment to a first container that holds the first fluid. The method can comprise connecting the second tube segment to a second container that holds the second fluid. The method can comprise connecting the third tube segment and/or the fourth tube segment to an administration tube, for example by a Y-connector.

Various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A flow set comprising:
a first one-way valve, a second one-way valve, a third one-way valve, and a fourth one-way valve;
the first one-way valve and the third one-way valve form at least a portion of a first flow path;
the second one-way valve and the fourth one-way valve form at least a portion of a second flow path;
at least one of the first, second, third and fourth one-way valves has a zero cracking pressure;
at least one of the first and third one-way valves has a cracking pressure greater than zero bar; and
at least one of the second and fourth one-way valves has a cracking pressure greater than zero bar.

2. The flow set of claim 1, wherein the first, second, third and fourth one-way valves have a set of cracking pressures selected from the group consisting of:
(i) each of the first and fourth one-way valves has a zero cracking pressure, and each of the second and third one-way valves has a cracking pressure greater than zero bar;
(ii) each of the first and fourth one-way valves has a cracking pressure greater than zero bar, and each of the second and third one-way valves has a zero cracking pressure;
(iii) each of the first and second one-way valves has a cracking pressure greater than zero bar, and each of the third and fourth one-way valves has a zero cracking pressure;
(iv) each of the third and fourth one-way valves has a cracking pressure greater than zero bar, and each of the first and second one-way valves has a zero cracking pressure; and
(v) only one of the first, second, third and fourth one-way valves has a zero cracking pressure, and three of the first, second, third and fourth one-way valves have a cracking pressure greater than zero bar.

3. The flow set of claim 1, wherein each of the first, second, third and fourth one-way valves is a slit valve.

4. The flow set of claim 1, comprising a first valve assembly that provides the first and second one-way valves, and a second valve assembly that provides the third and fourth one-way valves.

5. The flow set of claim 4, wherein the first one-way valve comprises a first flexible membrane in the first valve assembly, the second one-way valve comprises a second flexible membrane in the first valve assembly, the fourth one-way valve comprises a first flexible membrane in the second valve assembly, and the third one-way valve comprises a second flexible membrane in the second valve assembly.

6. The flow set of claim 5, wherein:
each of the first and second valve assemblies comprises an inlet port, a pump communication port, and an outlet port, and
each of the first and second valve assemblies comprises a chamber by which the inlet, pump communication, and outlet ports fluidly communicate with each other.

7. The flow set of claim 6, wherein the first flexible membrane of the first valve assembly is between the inlet port of the first valve assembly and the chamber of the first valve assembly, the first flexible membrane of the second valve assembly is between the inlet port of the second valve assembly and the chamber of the second valve assembly, the second flexible membrane of the first valve assembly is between the chamber of the first valve assembly and the outlet port of the first valve assembly, and the second flexible membrane of the second valve assembly is between the chamber of the second valve assembly and the outlet port of the second valve assembly.

8. The flow set of claim 6, comprising a connecting tube extending from the pump communication port of the first valve assembly to the pump communication port of the second valve assembly.

9. The flow set of claim 6, comprising a first tube segment connected to the inlet port of the first valve assembly, and the first tube segment comprises a portion of the first flow path.

10. The flow set of claim 9, comprising a second tube segment connected to the inlet port of the second valve assembly, and the second tube segment comprises a portion of the second flow path.

11. The flow set of claim 10, comprising a third tube segment connected to the outlet port of the first valve assembly, and the third tube segment comprises a portion of the second flow path.

12. The flow set of claim 11, comprising a fourth tube segment connected to the outlet port of the second valve assembly, and the fourth tube segment comprises a portion of the first flow path.

13. The flow set of claim 12, comprising a Y-connector that connects the third tube segment and the fourth tube segment to an administration tube.

14-16. (canceled)

17. A flow set comprising:
a first one-way valve, a second one-way valve, a third one-way valve, a fourth one-way valve, a fifth one-way valve upstream of the first one-way valve, and a sixth one-way valve upstream of the fourth one-way valve,
the fifth one-way valve, the first one-way valve, and the third one-way valve form at least a portion of a first flow path,
the sixth one-way valve, the second one-way valve, and the fourth one-way valve form at least a portion of a second flow path, and
each of the fifth and sixth one-way valves has a cracking pressure greater than zero bar.

18. The flow set of claim 17, wherein at least one of the first, second, third and fourth one-way valves has a cracking pressure of zero bar.

19-22. (canceled)

23. A flow set comprising:
a first one-way valve, a second one-way valve, a third one-way valve, a fourth one-way valve, and an additional one-way valve downstream of the second and third one-way valves;
the first one-way valve, the third one-way valve and the additional one-way valve form at least a portion of a first flow path;
the second one-way valve, the fourth one-way valve and the additional one-way valve form at least a portion of a second flow path; and
the additional one-way valve has a cracking pressure greater than zero bar.

24. The system of claim 23, wherein at least one of the first, second, third and fourth one-way valves has a cracking pressure of zero bar.

25-39. (canceled)
